(19) United States
(12) Patent Application Publication
(10) Pub. No.: US 2024/0216293 A1
(43) Pub. Date: Jul. 4, 2024

(54) USE OF PERILLYL ALCOHOL TO ENHANCE LEVO-DOPA DELIVERY

(71) Applicant: NEONC TECHNOLOGIES, INC, Los Angeles, CA (US)

(72) Inventor: Thomas CHEN, La Canada, CA (US)

(21) Appl. No.: 18/557,650

(22) PCT Filed: Apr. 28, 2022
(86) PCT No.: PCT/US22/26656

§ 371 Date: Oct. 27, 2023

Related U.S. Application Data
(60) Provisional application No. 63/180,936, filed on Apr. 28, 2021.

(51) Int. Cl. A61K 31/045; A61K 9/00; A61K 31/195; A61P 25/16

(52) U.S. Cl. CPC A61K 31/045; A61K 9/0043; A61K 31/195; A61P 25/16

(57) ABSTRACT

The present application presents compositions and methods for the intranasal administration of perillyl alcohol and L-Dopa to a patient in need of treatment for Parkinson's Disease. The compositions and methods of the present application enhance the entry of L-Dopa into the brain of the patient suffering from Parkinson's Disease

### CROSS REFERENCE TO RELATED APPLICATIONS

The present application claims priority to U.S. Patent Application Ser. No. 63/180,936 filed Apr. 28, 2021, which is hereby incorporated by reference in its entirety.

### FIELD OF THE INVENTION

This invention related to the use of perillyl alcohol (POH) to enhance Levo-Dopa (L-Dopa) delivery to a mammal. Specifically, the POH is used for patients in need of treatment for the management of Parkinson's disease (PD).

### BACKGROUND OF THE INVENTION

Parkinson's disease (“PD”) is a long-term degenerative disorder of the central nervous system that mainly affects the motor system. The symptoms usually emerge slowly and, as the disease worsens, non-motor symptoms become more common. The most obvious early symptoms are tremor, rigidity, slowness of movement, and difficulty with walking. Cognitive and behavioral problems may also occur with depression, anxiety, and apathy occurring in many people with PD. Parkinson's disease dementia becomes common in the advanced stages of the disease. Those with Parkinson's can also have problems with their sleep and sensory systems. The motor symptoms of the disease result from the death of cells in the substantia nigra, a region of the midbrain, leading to a dopamine deficit. The cause of this cell death is poorly understood but involves the build-up of misfolded proteins into Lewy bodies in the neurons.

Management of Parkinson's disease is a challenge that requires a tailored approach for each individual. In the advanced phase of the disease, patients may experience motor complications despite optimized pharmacological therapy. Apomorphine, a short-acting D₁- and D₂-like receptor agonist, has been shown to provide rapid and effective relief from unpredictable “off” periods. Apomorphine is an aporphine alkaloid derived from acidification of morphine. Its molecular formula is C₁₇H₁₇NO₂.

Perillyl alcohol (POH), a naturally occurring monoterpene, has been suggested to be an effective agent against a variety of cancers, including CNS cancer, breast cancer, pancreatic cancer, lung cancer, melanomas and colon cancer. Gould, M. Cancer chemoprevention and therapy by monoterpenes. Environ Health Perspect. 1997, 105 (Suppl 4): 977-979.

Intranasal delivery of a drug offers a non-invasive therapy to bypass the blood brain barrier and to rapidly deliver pharmaceutical agents to the CNS directly. Intranasally administered drugs reach the parenchymal tissues of the brain, spinal cord and/or cerebrospinal fluid (CSF) within minutes. In addition to delivery via the olfactory tract and trigeminal nerves, it appears from animal studies that the therapeutic drug is also delivered systemically through the nasal vasculature. Hashizume et al. New therapeutic approach for brain tumors: intranasal delivery of telomerase inhibitor GRN163. Neuro-oncology 10: 112-120, 2008. Thorne et al. Delivery of insulin-like growth factor-1 to the rat brain and spinal cord along olfactory and trigeminal pathways following intranasal administration. Neuroscience 127: 481-496, 2004. Intranasal delivery of therapeutic agents may provide a systemic method for treating other types of cancers, such as lung cancer, prostate cancer, breast cancer, hematopoietic cancer and ovarian cancer, etc. Wang et al. have demonstrated that intranasal delivery has allowed for delivery of blood brain barrier (BBB) impermeable medications such as bortezomib to the brain. (Wang-Journal of Neurosurgery)

### SUMMARY OF THE INVENTION

In some embodiments, the present invention provides for a method of administering L-Dopa (L-3,4-dihydrophenylalanine) to a central nervous system of a mammal (e.g., a human), the method comprising administering a pharmaceutical comprising a monoterpene concurrently with the L-Dopa.

In some embodiments, the central nervous system is the brain.

In some embodiments, the present invention provides for a method of administering L-Dopa to a mammal (e.g., a human), the method comprising administering a monoterpene concurrently with the L-Dopa.

In some embodiments, the present invention provides for a method of administering a pharmaceutical composition comprising a monoterpene and L-Dopa to a mammal (e.g., a human). In these embodiments, the monoterpene and L-Dopa are mixed to form a mixture. In some embodiments, the administration is an intranasal administration.

In some embodiments, the monoterpene may be perillyl alcohol.

In some embodiments, the present application provides for a pharmaceutical composition comprising perillyl alcohol that can be administered via intranasal application to enhance L-Dopa entry into the brain of a patient, wherein in some embodiments, the patient is in need of treatment for the management of PD.

In some embodiments, the present application provides for a pharmaceutical composition comprising perillyl alcohol and at least one pharmaceutically acceptable carrier or diluent that can be administered via intranasal application to enhance L-Dopa entry into the brain of a patient, wherein some embodiments, the patient is in need of treatment for the management of PD.

In some embodiments, the present application provides for a pharmaceutical composition comprising POH and L-Dopa that can be administered via intranasal application to enhance the entry of L-Dopa into the brain of a patient, wherein some embodiments, the patient is in need of treatment for the management of PD.

Perillyl alcohol has the following structure

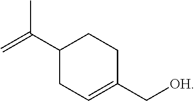

In some embodiments, the pharmaceutically acceptable carrier or diluent may be ethanol, glycerol, or a combination thereof.

In some embodiments, the invention further provides for a method for treating a disease in a mammal, comprising the step of delivering to the mammal a therapeutically effective amount of POH and a pharmaceutically acceptable carrier or diluent concurrently with the administering of L-Dopa into a mammal. In some embodiments, the diseases treated may be PD. The route of administration of the perillyl alcohol include intranasal delivery.

In some embodiments, the invention further provides for a method for treating a disease in a mammal, comprising the step of delivering to the mammal a therapeutically effective amount of a pharmaceutical composition comprising POH and L-Dopa into a mammal. The diseases treated may be PD. The route of administration of the pharmaceutical composition comprising POH and L-Dopa include intranasal. In some embodiments, the pharmaceutical composition also comprises a pharmaceutically acceptable carrier or diluent.

In some embodiments, the monoterpene may be a purified version of POH. This purified version of POH is sometimes referred to as NEO100. NEO100 is a highly purified (greater than about 99% pure) POH. The NEO100 can be made under current good manufacturing practices (GMP) conditions. In some embodiments, the monoterpene is provided in an amount in the range from about 0.01% (w/v) to about 5% (w/v), 0.01% (w/v) to about 1% (w/v), or 0.3 (w/v) of NEO100 in 50:50 ethanol:glycerol.

NEO100 has been used in Phase I/IIa trials in patients with recurrent malignant gliomas without toxic side effects (Schönthal A. H., et al. Phase I trial of intranasal NEO100, highly purified perillyl alcohol, in adult patients with recurrent glioblastoma. Neurooncol Adv. 2021 Feb. 12).

In some embodiments. NEO100 can be used with any drug that can dissolve in NEO100.

### BRIEF DESCRIPTION OF THE DRAWINGS

The FIG. shows the results from rotational tests after mice were treated with various compounds.

### DETAILED DESCRIPTION OF THE INVENTION

The present invention provides for methods of using a monoterpene to enhance L-Dopa delivery into a central nervous system of a mammal.

The monoterpene may have a purity of greater than about 98.5% (v/v), greater than about 99.0% (v/v), or greater than about 99.5% (v/v).

The monoterpene may be formulated into a pharmaceutical composition in the presence or absence of a carrier or diluent, where the monoterpene is present in amounts ranging from about 0.01% (w/v) to about 100% (w/v), from about 0.1% (w/v) to about 80% (w/v), from about 1% (w/v) to about 70% (w/v), from about 10% (w/v) to about 60% (w/v), from about 1% (w/v) to about 10% (w/v), from about 1% (w/v) to about 5% (w/v), from about 1% (w/v) to about 3% (w/v), from about 3% (w/v) to about 10% (w/v), or from about 0.1% (w/v) to about 20% (w/v). In some embodiments, where the monoterpene is present in amounts ranging from about 0.01% (w/v) to about 5% (w/v), about 0.01% (w/v) to about 1% (w/v), or in an amount of 0.3% (w/v).

The monoterpene and the L-Dopa may be administered concurrently. In some embodiments, the monoterpene and the L-Dopa are mixed to form a mixture prior to administration. They may exert an advantageously combined effect (e.g., additive or synergistic effects).

The route of administration may vary and can include intranasal.

The present invention also provides for a method of treating a disease such as treating the one or more symptoms of PD, comprising the step of delivering to a patient the present composition.

A specific example of a monoterpene that may be used in the present invention is perillyl alcohol (commonly abbreviated as POH). Perillyl alcohol compositions of the present invention can contain (S)-perillyl alcohol, (R)-perillyl alcohol, or a mixture of (S)-perillyl alcohol and (R)-perillyl alcohol.

In some embodiments, the monoterpene may be a purified version of POH, referred to as NEO100. NEO100 is a highly purified (>99%) POH. In some embodiments, the monoterpene is provided in an amount in the range from about 0.01% (w/v) to about 5% (w/v) or 0.01% (w/v) to about 1% (w/v) of NEO100 in 50:50 ethanol:glycerol. In some embodiments, the monoterpene is provided in an amount of 0.3% (w/v) of NEO100 in 50:50 ethanol:glycerol.

In the context of the present disclosure insofar as it relates to any of the disease conditions recited herein, the terms “treat,” “treatment,” and the like mean to relieve or alleviate at least one symptom associated with such condition, or to slow or reverse the progression of such condition. Within the meaning of the present disclosure, the term “treat” also denotes to arrest, delay the onset (i.e., the period prior to clinical manifestation of a disease) and/or reduce the risk of developing or worsening a disease.

The phrase “pharmaceutically acceptable,” as used in connection with compositions and/or cells of the present disclosure, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions and/or cells to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. In some embodiments, Pharmaceutically acceptable carriers may include ethanol, glycerol, or a combination thereof. Sec, e.g. Remington: The Science and Practice of Pharmacy 20th Ed. (2000) Lippincott Williams and Wilkins, Ed. K. E. Hoover.

To prepare such pharmaceutical compositions, one or more of the monoterpenes and/or at least one therapeutic agent may be mixed with a pharmaceutical acceptable carrier, adjuvant and/or excipient, according to conventional pharmaceutical compounding techniques. The therapeutic agent can be L-Dopa. Pharmaceutically acceptable carriers that can be used in the present compositions encompass any of the standard pharmaceutical carriers, such as a phosphate buffered saline solution, water, and emulsions, such as an oil/water or water/oil emulsion, and various types of wetting agents. The compositions can additionally contain solid pharmaceutical excipients such as starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, particularly for injectable solutions, include water, saline, aqueous dextrose, and glycols. For examples of carriers, stabilizers and adjuvants, see Remington's Pharmaceutical Sciences, edited by E. W. Martin (Mack Publishing Company, 18th ed., 1990). The compositions also can include stabilizers and preservatives.

In some embodiments, the L-Dopa can be administered may be administered at a dose ranging from about 0.050 mg/kg to about 500 mg/kg of body weight. Other ranges, include, about 0.1 mg/kg to about 100 mg/kg, about 1 mg/kg to about 50 mg/kg, about 5 mg/kg to about 35 mg/kg, and about 10 mg/kg to about 30 mg/kg. In some embodiments, the L-Dopa can be administered at a dose of 30 mg/kg.

As used herein, the term “therapeutically effective amount” is an amount sufficient to treat a specified disorder or disease or alternatively to obtain a pharmacological response treating a disorder or disease. Methods of determining the most effective means and dosage of administration can vary with the composition used for therapy, the purpose of the therapy, the target cell being treated, and the subject being treated. Treatment dosages generally may be titrated to optimize safety and efficacy. Single or multiple administrations can be carried out with the dose level and pattern being selected by the treating physician. Suitable dosage formulations and methods of administering the agents can be readily determined by those of skill in the art.

The present disclosure also provides the compositions as described above for intranasal administration. As such, the compositions can further comprise a permeation enhancer. Southall et al. Developments in Nasal Drug Delivery, 2000. The present compositions may be administered intranasally in a liquid form such as a solution, an emulsion, a suspension, drops, or in a solid form such as a powder, gel, or ointment. Devices to deliver intranasal medications are well known in the art. Nasal drug delivery can be carried out using devices including, but not limited to, intranasal inhalers, intranasal spray devices, atomizers, nasal spray bottles, unit dose containers, pumps, droppers, squeeze bottles, nebulizers, metered dose inhalers (MDI), pressurized dose inhalers, insufflators, and bi-directional devices. The nasal delivery device can be metered to administer an accurate effective dosage amount to the nasal cavity. The nasal delivery device can be for single unit delivery or multiple unit delivery. In a specific example, the ViaNase Electronic Atomizer from Kurve Technology (Bethell, Washington) can be used in this invention (http://www.kurvetech.com). The compounds of the present invention may also be delivered through a tube, a catheter, a syringe, a packtail, a pledget, a nasal tampon or by submucosal infusion. U.S. Patent Publication Nos. 20090326275, 20090291894, 20090281522 and 20090317377.

The present compositions can be formulated as aerosols using standard procedures. The monoterpene and/or at least one therapeutic agent may be formulated with or without solvents, and formulated with or without carriers. The formulation may be a solution, or may be an aqueous emulsion with one or more surfactants. For example, an aerosol spray may be generated from pressurized container with a suitable propellant such as, dichlorodifluoromethane, trichlorofluoromethane, dichlorotetrafluoroethane, hydrocarbons, compressed air, nitrogen, carbon dioxide, or other suitable gas. The dosage unit can be determined by providing a valve to deliver a metered amount. Pump spray dispensers can dispense a metered dose or a dose having a specific particle or droplet size. As used herein, the term “aerosol” refers to a suspension of fine solid particles or liquid solution droplets in a gas. Specifically, aerosol includes a gas-borne suspension of droplets of a monoterpene, as may be produced in any suitable device, such as an MDI, a nebulizer, or a mist sprayer. Aerosol also includes a dry powder composition of the composition of the instant invention suspended in air or other carrier gas. Gonda (1990) Critical Reviews in Therapeutic Drug Carrier Systems 6: 273-313. Raeburn et al., (1992) Pharmacol. Toxicol. Methods 27: 143-159.

The present compositions may be delivered to the nasal cavity as a powder in a form such as microspheres delivered by a nasal insufflator. The present compositions may be absorbed to a solid surface, for example, a carrier. The powder or microspheres may be administered in a dry, air-dispensable form. The powder or microspheres may be stored in a container of the insufflator. Alternatively, the powder or microspheres may be filled into a capsule, such as a gelatin capsule, or other single dose unit adapted for nasal administration.

The pharmaceutical composition can be delivered to the nasal cavity by direct placement of the composition in the nasal cavity, for example, in the form of a gel, an ointment, a nasal emulsion, a lotion, a cream, a nasal tampon, a dropper, or a bioadhesive strip. In certain embodiments, it can be desirable to prolong the residence time of the pharmaceutical composition in the nasal cavity, for example, to enhance absorption. Thus, the pharmaceutical composition can optionally be formulated with a bioadhesive polymer, a gum (e.g., xanthan gum), chitosan (e.g., highly purified cationic polysaccharide), pectin (or any carbohydrate that thickens like a gel or emulsifies when applied to nasal mucosa), a microsphere (e.g., starch, albumin, dextran, cyclodextrin), gelatin, a liposome, carbamer, polyvinyl alcohol, alginate, acacia, chitosans and/or cellulose (e.g., methyl or propyl; hydroxyl or carboxy; carboxymethyl or hydroxylpropyl).

Nebulizer devices produce a stream of high velocity air that causes a therapeutic agent in the form of liquid to spray as a mist. The therapeutic agent is formulated in a liquid form such as a solution or a suspension of particles of suitable size. In one embodiment, the particles are micronized. The term “micronized” is defined as having about 90% or more of the particles with a diameter of less than about 10 μm. Suitable nebulizer devices are provided commercially, for example, by PARI GmbH (Starnberg, Germany). Other nebulizer devices include Respimat (Bochringer Ingelheim) and those disclosed in, for example, U.S. Pat. Nos. 7,568,480 and 6,123,068, and WO 97/12687. The monoterpenes can be formulated for use in a nebulizer device as an aqueous solution or as a liquid suspension.

The device for intranasal administration may be an intranasal spray device, an atomizer, a nebulizer, a metered dose inhaler (MDI), a pressurized dose inhaler, an insufflator, an intranasal inhaler, a nasal spray bottle, a unit dose container, a pump, a dropper, a squeeze bottle, or a bi-directional device.

In some embodiments, the composition of the present invention can be delivered through a nasal spray applicator. If intra-nasal application is desired, the composition may be placed in an intra-nasal spray-dosing device or atomizer and then, be applied by spraying it into the nostrils of a patient for delivery to the mucous membrane of the nostrils. A sufficient amount is applied to achieve the desired systemic or localized drug levels. Intranasal sprays deliver about 5 cc microliters, with 1 cc-10 cc microliters being typically applied. In some embodiments, there is about 5 cc microliters per dose. One or more nostrils may be dosed and the application may occur as often as desired or as often as is necessary.

In some embodiments, the spray composition of the invention is generally employed in a dosing regimen that is dependent on the patient being treated. The frequency of use and the amount of the dose may vary from patient to patient. The patient may receive multiple doses during the day. One skilled in the art such as a physician can select the dosing regimen and dosage for a particular patient or patients.

Dosing is independent of how far the intra-nasal spray device is inserted into the nostril, whether the patient is inspiring or the angle of insertion of the device.

PD impairs the motor control of the body and is the result of the selective death of dopaminergic (DA) neurons in the substantia nigra of the midbrain. The disease is characterized by the aggregation of α-synuclein to form Lewy bodies in the neuron. Many reports have shown a link between toxin exposure and raised risk of PD. The 6-hydroxydopamine (6-OHDA) is a specific neurotoxin which targets catecholamine neurons through the dopamine active transporter (DAT). When 6-OHDA is injected into the median forebrain bundle or into the neostriatum of brain, it causes an irreversible loss of DA neurons in the ventral midbrain. The consistent loss of dopamine innervation in target areas is linked with a range of long-term, behavioral deficits. Thus, a 6-OHDA-induced lesion is the most widely used animal model of PD.

The following example is presented for the purposes of illustration only and is not limiting the invention.

### Example

### Inducing Unilateral Lesions of PD in C57BL/6 Mice by Injection of 2,4,5-Trihydrophenethylamine (“6-OHDA”)

C57BL/6 mice were sedated and placed in a stereotaxic frame. 12 ug of 6-OHDA was injected into the right Substantial Nigra pars reticulata (SNpc) of the DA pathway, creating hemi-parkinsonian mice. The 6-OHDA was injected into the mice brain according to following coordinates: 3 mm caudal to bregma; 1.2 mm lateral to midline: 4.8 mm ventral to the dural surface; and the tooth-bar will be set at 1.0 mm below the interaural line. The speed of 6-OHDA injection was 1.0 ul/min, and the cannula was left in situ for an additional 4 min after the completion of injection before its withdrawn.

### The Confirmation of PD in Mice Via Apomorphine Rotation Test

To verify successful lesion formation, 3 weeks after the 12 μg 6-OHDA injection, an apomorphine rotation test was given.

First, rotational behavior without apomorphine was tested. The mice were placed into a test apparatus and full body ipsilateral (towards the lesion side) and contralateral side (away from the lesion side) rotations were manually counted for 5 min after a short habituation period. The mice were all hemiparetic.

Then rotational behavior with apomorphine was tested. The mice were given a subcutaneous injection to the scruff of the neck of apomorphine. The injection of apomorphine was 0.5 mg/kg body weight subcutaneously with 0.2% ascorbic acid and 0.9% saline solution. The mice were again placed into the test apparatus. After 10 min, rotational behavior was assessed for a 60-min period. The full body ipsilateral and contralateral side rotations were manually counted.

The away from the lesion rotations were counted, and more than 50 turns per minute were considered to demonstrate a positive effect after PD was induced.

### Intranasal NEO100 Facilitated L-Dopa Delivery.

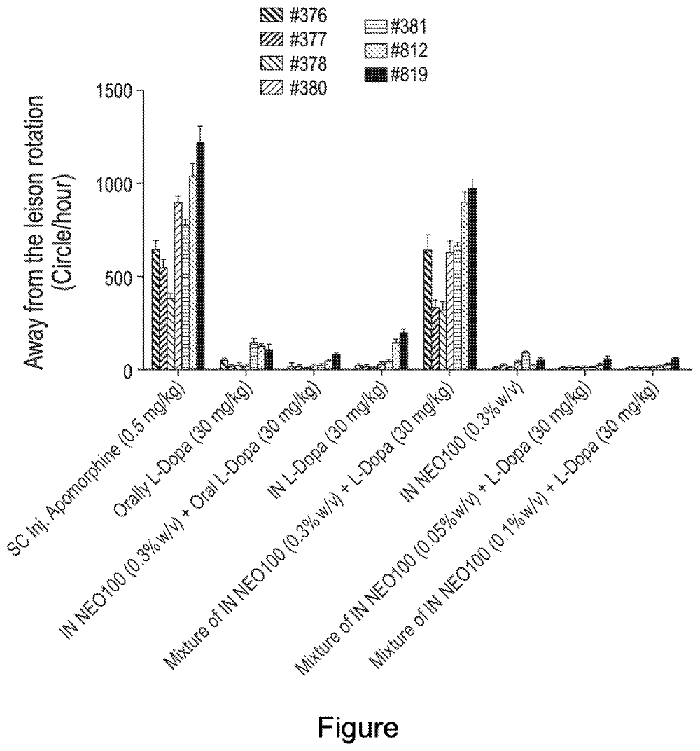

FIG. 1 illustrates data from seven experimental mice (mouse #376, mouse #377, mouse #378, mouse #380, mouse #381, mouse #812, and mouse #819) that were confirmed with positive PD after challenged by subcutaneous injection of apomorphine, as described above.

The seven mice were then tested using varied treatment regimens on different days as described previously. The varied treatment methods include (1) oral L-dopa, (2) intranasal (“IN”) NEO100 (0.3% w/v) and Oral L-Dopa, (3) IN L-dopa, (4) IN of a mixture of NEO100 (0.3% w/v) and L-Dopa, (5) IN NEO100 (0.3% w/v), (6) a mixture of NEO100 (0.05% w/v/) and L-Dopa, and (7) a mixture of NEO100 (0.1% w/v) and L-Dopa. In each of these varied treatment methods, the dose of oral L-dopa was fixed at 30 mg/kg.

The results indicate that IN NEO100 (0.3% w/v) and Oral L-Dopa induces the mice to make contralateral rotations away from lesions caused by 6-OHDA injection in the medial forebrain bundle, MFB.

### Setup of Rotation Station

Plastic cylinder: The cylinder has a 12.5 inch-diameter and was placed on a flat surface on top of a single sheet of construction paper. The side of the cylinder was coated with construction paper.

Red Light: A red-light was mounted above the cylinder, allows the visualization of the mouse in the dark, which is the mouse's active cycle. However, the mouse itself is unable to detect red color very well, so the mouse perceived its environment as dark, keeping it active.

Video Camera: A video camera was mounted above the cylinder. The entire platform mouse was rotating on was visible and well lit. The camera used must have a revolving screen. This screen was used to visualize and count rotations of the mice. Rotations were also be recorded.

Counting method and rest periods: The mice were monitored for 60 min, and the net contralateral the lesion rotations were counted. The net contralateral rotations were calculated by subtracting (total right-total left 360° turns) per minute were counted. Mice were given at least 4 days following each apomorphine dose to ensure that the drugs had been eliminated from the system prior to behavioral testing.

### Statistical Analyses

A two-tailed student t test was used. The log-rank test will also be used to evaluate significance for the L-dopa initiated ipsilateral rotation to the lesion side. P<0.05 will be considered significant. The calculated p value in individual tested mouse, #376, #377, #378, #380, #381, #812, and #819 is**0.0058 vs.**0.0049;**0.0033 vs.**0.003;**0.0035 vs.**0.0044;*0.035 vs.*0.027;***0.00067 vs.***0.00011;**0.008 vs.**0.0011; and**0.0026 vs.**0.0024, respectively.

Therefore, since intranasal delivery of a mixture of NEO100 and L-Dopa induces contralateral rotations, the mixture could be suitable for treatment to provide relief from unpredictable “off” periods in PD patients.

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description and accompanying drawings is offered by way of illustration only and not as a limitation.

1. A pharmaceutical composition comprising perillyl alcohol (POH) and L-Dopa,
wherein the pharmaceutical composition is formulated for intranasal delivery to a mammal.

2. The pharmaceutical composition of claim 1, wherein the composition comprises perillyl alcohol in an amount in the range of from about 0.01% (w/v) to about 5% (w/v).

3. The pharmaceutical composition of claim 1, wherein the composition comprises L-Dopa in an amount in the range of from about 5 mg/kg to about 35 mg/kg of body weight of a subject.

4. The pharmaceutical composition of claim 1, further comprising one or more pharmaceutically acceptable carrier or diluent.

5. The pharmaceutical composition of claim 4, wherein the one or more pharmaceutically acceptable carrier or diluent is an ethanol/glycerol mixture, wherein the mixture has a ratio of 1:1 ethanol-to-glycerol.

6. A method of treating one or more symptoms in a patient that has Parkinson's Disease, the method comprising:
administering a pharmaceutical composition comprising POH to the patient; and
administering L-Dopa to the patient,
wherein the POH is administered concurrently with the L-Dopa.

7. A method of treating one or more symptoms in a patient that has Parkinson's Disease, the method comprising:
administering a pharmaceutical composition to a patient, the pharmaceutical composition comprising POH and L-Dopa.

8. The method of claim 6, wherein the pharmaceutical composition further comprises one or more pharmaceutically acceptable carrier or diluent.

9. The method of claim 6, wherein the pharmaceutical composition is administered intranasally.

10. The method of claim 6, wherein the one or more pharmaceutically acceptable carrier or diluent is an ethanol/glycerol mixture, wherein the mixture has a ratio of 1:1 ethanol-to-glycerol.

11. The method of claim 7, wherein the pharmaceutical composition further comprises one or more pharmaceutically acceptable carrier or diluent.

12. The method of claim 7, wherein the pharmaceutical composition is administered intranasally.

13. The method of claim 7, wherein the one or more pharmaceutically acceptable carrier or diluent is an ethanol/glycerol mixture, wherein the mixture has a ratio of 1:1 ethanol-to-glycerol.
